(19) United States
(12) Patent Application Publication
(10) Pub. No.: US 2011/0146694 A1
(43) Pub. Date: Jun. 23, 2011

(54) PATIENT EXTREMITY DRAPE

(76) Inventors: Michael Fischer, Pettneu (AT); Karin T. Slootjes, Roermond (NL); Gerrit P. Driesssen, Siebengewald (NL)

(21) Appl. No.: 12/747,192

(22) PCT Filed: Dec. 17, 2008
(86) PCT No.: PCT/US2008/087146

§ 371 Date: Mar. 8, 2011

(30) Foreign Application Priority Data
Dec. 17, 2007 (GB) 0724517.8

(51) Int. Cl. A61B 19/12

(52) U.S. Cl. 128/856

(57) ABSTRACT

A drape for covering a patient extremity. The drape comprises (a) a flexible tube having at least one open end and (b) an elongated bandage. At least one end portion of the elongated bandage is permanently affixed in a position on the outer surface of the tube.

### FIELD

The present invention relates to drapes adapted to cover a patient extremity during operative procedures, including e.g. surgical procedures.

### BACKGROUND

In surgical procedures it is desirable and/or necessary to isolate the area of the incision from surrounding areas in order to reduce the risk of contamination and infection. Generally this entails covering essentially the entire patient outside the operative sites, including extremities, such as arms and legs. The same holds true for a number of other non-surgical operative procedures.

Sleeves made of an elastic knitted material, i.e. stockinettes, can be used to cover extremities. However the use of such sleeves can be ineffective due to poor barrier characteristics (e.g. allowing migration of bacteria), once the stockinette becomes wet with blood or other liquids during an operative procedure. So-called impervious stockinettes made of an inner layer of elastic knitted material joined to an outer layer made of a moisture or liquid-proof film show improved effectiveness in regard to blocking bacterial migration. However such impervious stockinettes are relative expensive and often very difficult for medical personnel to put on, in particular to put on in an aseptic manner, where normally at least two people (e.g. at least two nurses) are required to place one such stockinette onto an extremity.

Patient extremity drapes that do not include a stockinette are known. Such drapes conventionally comprise a flexible liquid-impervious material and have a tubular form that typically is inelastic and shaped and sized so as to loosely fit around the extremity (e.g. not form-fitting). Such patient extremity drapes generally comprising a nonwoven or a nonwoven-containing composite or laminate material are typically more cost-effective and for the most part disposable. Examples of such patient extremity drapes may be found in among others U.S. Pat. Nos. 3,989,040 (Lofgren, Farrow); 3,494,356 (Melges) and U.S. Pat. No. 3,769,971 (Collins).

U.S. Pat. No. 3,989,040 discloses a disposable patient extremity drape made of a liquid-proof film bonded to a nonwoven, where the drape is provided with adhesive strip(s) for securing the drape directly to patient to reduce the risk of the drape being dislodged during surgical procedures.

U.S. Pat. No. 3,494,356 discloses nonwoven leggings including unitary ties and/or adhesive for fastening the leggings, an abdominal drape and an underbuttocks drape to each other.

### SUMMARY

There is an ongoing problem of such patient extremity drapes moving, slipping or falling off during operative procedures, even such drapes provided with adhesive strips for securing the drape directly to the patient or to another drape. This problem is particularly acute when the extremity is moved (e.g. rotated) during an operative procedure. Also some patients have allergic reactions to adhesives making it undesirable to directly fasten an extremity drape onto the patient using an adhesive. Previous attempts to secure such extremity drapes with other fasteners, such as unitary ties, have been largely unsuccessful and/or have been found to be cumbersome, e.g. often requiring a concerted effort of several people to drape and secure one extremity of the patient, in particular when the extremity to be draped has considerable weight, such as a lower limb.

Surprisingly by providing a patient extremity drape including a flexible tube with an elongated bandage, where one end of the elongated bandage is permanently affixed to the tube and positioned on the outer surface of the tube, it is possible to provide a drape which is easy to use while at the same time providing a stable draping showing a reduced tendency towards moving, slipping or falling off during an operative procedure.

Accordingly the present invention provides a drape for covering a patient extremity comprising
a) a flexible tube having a first open end, a second end and an outer surface, and
b) an elongated bandage having a first end portion,
wherein the first end portion of the elongated bandage is permanently affixed to the tube and wherein said first end portion of the elongated bandage is positioned on the outer surface of the tube.

The term “elongated bandage” as used herein is generally understood to mean that the bandage is sufficiently elongated so as to be capable of being wound 2 turns or more (more suitably 5 turns or more) about an extremity of a patient.

Desirably the second end of the tube is closed.

In use the patient extremity is inserted into the tube through the first open end and any excess draping material of the tube in its width can be folded or flapped over. Then the elongated bandage of the tube is wrapped about the extremity, stabilizing the draping and, thus, facilitating a reduced tendency towards moving, slipping or falling off during an operative procedure, whereby the provision of the elongated bandage having one end securely affixed to the tube facilitates easy use by medicinal personnel.

To provide enhanced draping stability and ease in use, in particular ease in use by one person (e.g. a nurse) to drape and secure an extremity of the patient, preferably the first end portion of the elongated bandage is positioned on the outer surface of the tube near the second end of the tube (i.e. spaced apart from the second end, but closer to (and thus close to) the second end of the tube) or adjacent to the second end of the tube. In use of such preferred drapes, again the patient extremity is inserted into the tube through the first open end, any excess draping material of the tube in its width can be folded or flapped over, and then the elongated bandage can be wrapped about the extremity generally in a direction up towards the open end of the tube. The provision of the elongated bandage with its first end securely affixed to the tube element of the drape near or adjacent to the second end of the tube (i.e. the end opposite to the open end into which the extremity is inserted) facilitates overall ease in use, for example generally allowing one person (e.g. a nurse) to easily lift and maneuver the extremity, even an extremity having considerable weight, while wrapping the bandage about the extremity. Furthermore, once the extremity is thus draped and wrapped, an advantageously stable draping is realized having a yet even further reduced tendency towards moving, slipping or falling off.

Favorably, patient extremity drapes described herein are provided sterilized, folded and/or rolled up, and sealed in a pouch ready for use.

It is noted that U.S. Pat. No. 4,679,552 discloses a sleeve for arthroscopic surgery including a wrap. However the wrap is not permanently affixed to the sleeve. Also the disclosed sleeve includes traction strips mounted within the tubular sleeve composed of a knit stockinette inner layer and latex outer layer and joined to the sleeve and a strap, said traction strips frictionally engaging the arm of the patient, and it is disclosed that with the described arrangement of the traction strips, strap and wrap, applied traction force is distributed over a broad area so as to avoid injury of the limb.

### BRIEF DESCRIPTION OF THE DRAWINGS

The present invention will be further explained with reference to the attached figures, wherein like structure or system elements are referred to by like reference numerals throughout the several views.

FIG. 1 shows a top view of an exemplary drape in accordance with the invention.

FIG. 2 shows a perspective view of the exemplary drape shown in FIG. 1.

FIG. 3 shows a top view of an alternative exemplary drape in accordance with the invention.

FIG. 4 shows a perspective view of the exemplary drape shown in FIG. 3.

FIG. 5 show a cross-sectional view of the drapes shown in FIG. 1 and FIG. 3 along lines 5-5.

FIG. 6 shows a perspective view of yet another exemplary drape in accordance with the invention.

FIG. 7a shows a top view of the exemplary drape shown in FIG. 3 where the elongated bandage is extended along its length in a single direction to lay flat, and

FIGS. 7b and 7c each show a top view of further exemplary drapes where the elongated bandage is extended along its length in a single direction to lay flat.

FIG. 8 shows the exemplary drape shown in FIG. 3 in use.

While the above-identified figures set forth illustrative embodiments of the present invention, other embodiments are also contemplated, as noted in the disclosure. In all cases, this disclosure presents the invention by way of representation and not limitation. It should be understood that numerous other modifications and embodiments can be devised by those skilled in the art which fall within the scope and spirit of the principles of this invention.

### DETAILED DESCRIPTION

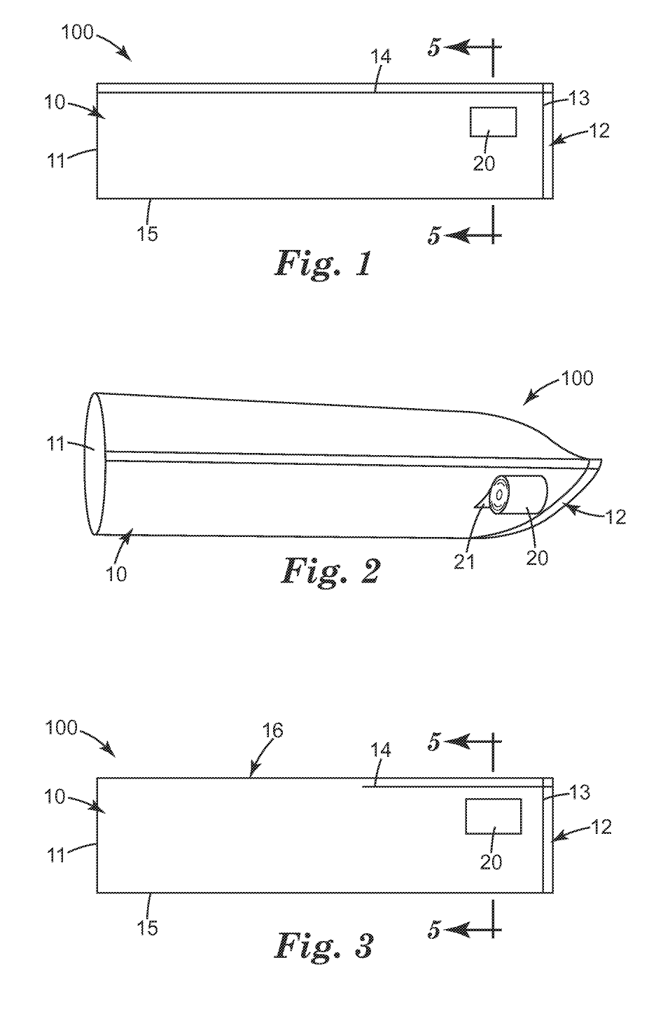

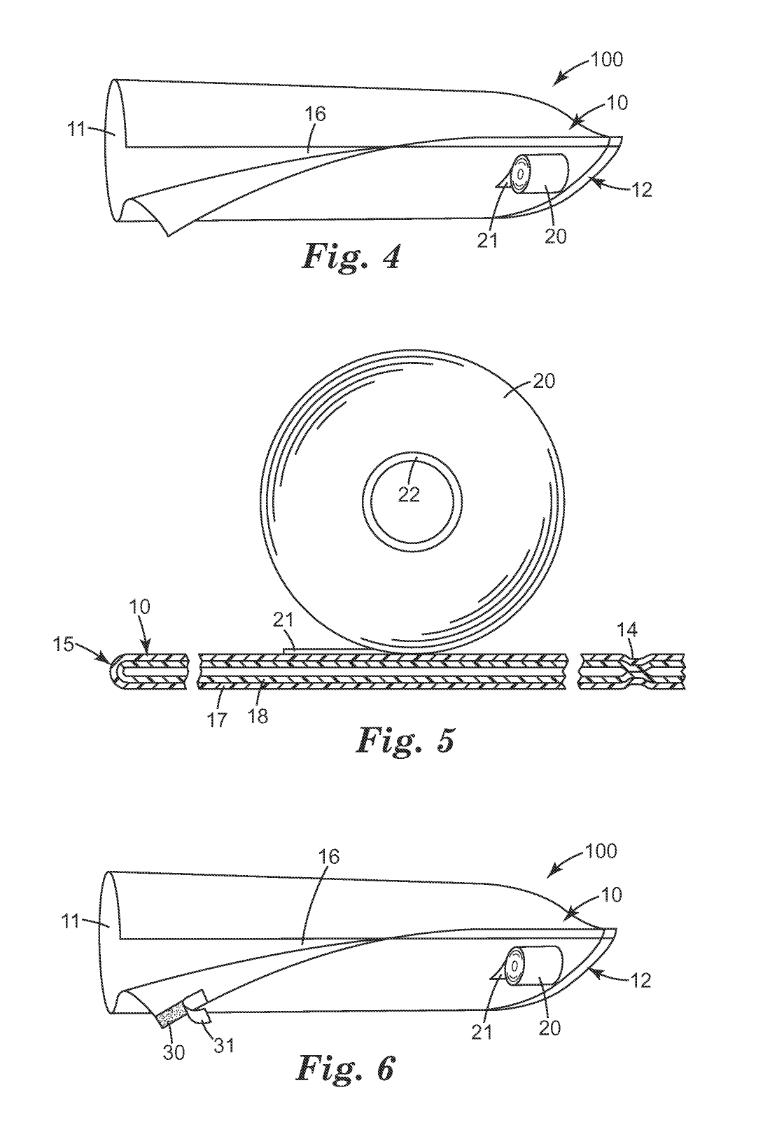

Patient extremity drapes in accordance with the present invention comprise a flexible tube having a first open end and a second end. This can be best understood by reference to FIGS. 1 to 4, providing different views of two alternative, exemplary drapes (100) in accordance with the present invention. In particular FIGS. 1 and 3 each show a top view of an exemplary drape (100) where the tube component (10) of the drape is unfolded or unrolled, lying flat, while FIGS. 2 and 4, each show a perspective view of the exemplary drape (100) shown in FIGS. 1 and 3, respectively. Referring to the illustrations, the tube (10) of the exemplary embodiments includes a first end (11) which is open for insertion of the extremity and a second end (12) which is favorably closed.

The tube may be made from a flexible material manufactured in a double open-ended tubular form (e.g. a polymeric film), wherein for favorable embodiments including a second closed end, the edges of one end are sealed. Alternatively, as can be appreciated from the illustrated exemplary embodiments, tube may be made (and is typically made) from a base sheet of a flexible material. The base sheet, typically having a substantially rectangular form, is folded in half along a longitudinal fold line and then the appropriate edges or portions of edges of the folded sheet are sealed. Edges may be sealed through any suitable method providing a liquid impervious seam, such as thermal bonding or welding, ultrasonic welding, stitching, adhesive bonding, and if desired the relevant edges may be folded over on themselves to provide reinforced seams. In the exemplary embodiments shown in FIGS. 1 and 3, a pair of transverse edges is sealed along line 13, for example by thermal or ultrasonic welding, to provide a closed end (12). In the exemplary embodiment shown in FIG. 1 the pair of longitudinal edges opposite to the folded edge (15) is sealed, again for example by thermal or ultrasonic welding, along its entire length along line 14. In the exemplary embodiment shown in FIG. 3, only a portion of the pair of longitudinal edges opposite to the folded edge (15) is sealed along line 14 so as to provide a slit (16) extending from the first open end (11) partially along the length of tube towards the second closed end (12), which can be better seen in the perspective view shown in FIG. 4.

Generally it is favorable to provide a tube including a slit extending from first open end partially along the length of the tube towards the second end to facilitate insertion of the extremity into the tube, adjustment of length of the tube to the particular patient as well as the removal of air during draping to facilitate provision of a desirable sterile field and close fit of the extremity drape. In particular for ease in insertion of the extremity into the tube by a single person and/or removal of air during draping, it has been found desirable to provide a slit having a length relative to the total length of the tube, such that the ratio of length of the tube to length of the slit is 3:1 or less, more desirably 2:1 or less, and most desirably 1.67:1 or less. (In other words, the length of the slit is desirably at least 33% of the total length of the tube, more desirably at least 50% of the total length of the tube, and most desirably at least 60% of the total length of the tube.) Within these ranges, desirably the ratio of length of the tube to length of the slit is 1.25:1 or more, more desirably 1.33:1 or more, and most desirably 1.42:1 or more. (In other words, desirably the length of the slit is at most 80% of the total length of the tube, more desirably at most 75% of the total length of the tube, and most desirably at most 70% of the total length of the tube.)

Referring to an alternative exemplary embodiment shown in FIG. 6, desirable embodiments of extremity drapes including a slit as described herein, may be favorably provided with adhesive (30) on the inner surface of the tube, where the adhesive, typically in the form of an adhesive strip, extends along and is positioned adjacent to at least a portion of one edge of the slit. Such adhesive can be used to secure any excess draping material of the tube after insertion of the extremity into the tube by e.g. pulling the flap of draping material including the adhesive across and over the front of the tube and pressing the adhesive onto the outer surface of the tube. As can be appreciated from the exemplary embodiment shown in FIG. 6, desirably adhesive, again typically in the form of an adhesive strip, is adjacent to the top edge of the tube (i.e. the edge defining its open end) extending towards the other end of the tube. Adhesive may extend along only a portion of the edge of the slit or continuously or intermittently along entire length of the edge of the slit. To facilitate the removal of air through the slit during draping, desirably the adhesive extends along only a portion of the edge of the slit or intermittently along the length of the slit. Any suitable adhesive may be used. However to facilitate a routine detachment of the secured draping material, and thus easy removal of the extremity drape, after the completion of an operative procedure, releasable adhesives are favored, more particularly re-positionable adhesives to further, favorably allow re-positioning of the secured excess draping material if needed. The adhesive may be covered with a protective release liner, as can be appreciated from the exemplary embodiment shown in FIG. 6 showing a protective release liner (31) partially peeled away from the adhesive (30). Alternatively when a re-positionable adhesive is used, the extremity drape may be folded or rolled and packaged ready for use, such that the outer surface of the re-positionable adhesive faces the inner surface of the tube, in particular the underside of opposite slit edge, so that the adhesive is protected, without the need of a protective release liner.

It will be appreciated that the overall size, e.g. length and width, as well as overall shape of the tube can vary provided it can accommodate the dimensions, e.g. width and length, of the extremity to be draped. To facilitate e.g. ease of insertion of the extremity into the tube, favorably the tube is not form-fitting (e.g. the tube is shaped and sized as to loosely fit around the extremity to be draped). Favorably, the tube is inelastic or substantially inelastic. The tube component can, for example, include a uniform cylindrical shape or have a tapered configuration. In terms of varying size, for draping an adult human leg, for example, the tube may have a length from about 60 cm to about 115 cm and a width from about 30 cm to about 50 cm. It will be appreciated that the mentioned “width” typically refers the width to the tube as it is lying flat or substantially flat, whereby the circumference of the tube is then typically twice the width. It will be also appreciated that extremity drapes for a particular indication, e.g. draping an adult human leg, can be provided in a variety of size selections, e.g. extra small, small, medium, large and extra-large, etc., where e.g. a small adult human leg extremity drape could have a tube size of about 30 cm by about 75 cm and a large adult leg extremity drape could have a tube size of about 38 cm by about 96 cm. For applications in veterinary medicine, depending on the particular animal patient, appropriate, suitable dimensions may be larger (e.g. for equine extremity draping) or smaller (e.g. for canine extremity draping).

The tube can be composed of any flexible material suitable for use in an operation environment and creates a sterile barrier. Desirably the tube comprises a liquid impervious material. Such a liquid impervious material may be breathable, if desired and/or deemed necessary. A wide variety of flexible materials readily available in the field can be used for the tube. Suitable flexible materials include, but are not limited to, woven and nonwoven medical fabrics; laminated and/or intermittently bonded multi-layered materials; polymeric materials; repellant nonwoven materials; absorbent materials; web reinforced plastic films and combinations thereof. To provide a combination of desirable properties (e.g. liquid imperviousness together with liquid absorption and/or wear comfort for the patient, etc.), the tube is favorably made of a composite material comprising at least two layers, more favorably at least three layers. Individual layers may be either laminated (fully bonded) to adjacent layer(s), or intermittently bonded or peripherally bonded or a combination of different bonding may be used, e.g. two layers may be laminated to each other, while a third layer is peripherally bonded. Layers of such composite materials are generally coextensive, however they need not be. Favorably the tube comprises a nonwoven, in particular a nonwoven-containing composite. Nonwovens that can be used include spun lace, meltblown and spunbond nonwoven as well as bonded, carded nonwovens. Nonwoven-containing laminates that can be used include nonwoven-laminates such as spunbond-meltblown-spunbond (SMS) materials, as well as other layer arrangements, such as SMMS and SM. Other nonwoven-containing laminates that can be used include nonwoven-film laminates, such as SF (spunbond-film) and SFS laminates. A film in such nonwoven-film laminates may be a film preventing penetration of liquids (a liquid impervious film) or a microporous film preventing penetration of liquids but allowing breathability (a breathable, liquid impervious film). For example, referring to FIG. 5 showing a cross-section of the exemplary embodiments shown in FIGS. 1 and 3, the tube can be composed on an inner comfort layer (18) (e.g. made of a nonwoven, such as a spunbond) laminated to an outer liquid impervious layer (17) (e.g. made of a plastic film, such as an extruded polyethylene film). Alternative embodiments may include additional layers, for example the inner comfort layer may be a multilayer nonwoven laminate, such as a SM, a SMS or a SMMS laminate. Other alternative embodiments including three or more layers may include: an outer liquid absorbing layer (e.g. made of a nonwoven or a nonwoven laminate), an inner comfort layer (e.g. made of a nonwoven or a nonwoven laminate) with a liquid impervious layer sandwiched between the inner and outer layers.

While, if desired, a stockinette may be used under an extremity drape as described herein, favorably the drape itself does not comprise an integral, elastic, knitted stockinette.

As described above, patient extremity drapes in accordance with the present invention comprise an elongated bandage having a first end portion, wherein the first end portion of the elongated bandage is permanently affixed to the tube and wherein said first end portion of the elongated bandage is positioned on the outer surface of the tube. In preferred embodiments, the first end portion of elongated bandage is positioned near or adjacent to the second end of the tube. Again this can be best understood by reference to the two exemplary embodiments illustrated in FIGS. 1 to 4. In particular referring to the perspective views of the exemplary embodiments shown in FIGS. 2 and 4, it can be appreciated that the exemplary drapes (100) are provided with an elongated bandage (20) the first end portion (21) thereof positioned on the outer surface of the tube (10) near the second closed end (12). Desirably the first end portion of elongated bandage (e.g. the centerline of the first end portion in its width) is positioned from the second end at a distance equal to or less than (including down to zero) than that corresponding to the underlying position of the outer major joint (e.g. ankle or wrist joint) of the extremity, when the extremity is inserted into the drape. For example, for a medium size adult human leg extremity drape, where the tube size may be about 34 cm by about 85 cm, the first end portion of the elongated bandage (e.g. the centerline of the first end portion in its width) may be favorably positioned at a distance of about 12 cm or less (including down to 0 (zero) cm) from the second end (e.g., from the closed end) of the tube. For a medium size adult human arm extremity drape, where the tube size may be about 25 cm by about 70 cm, the first end portion of the elongated bandage (e.g. the centerline of the first end portion in its width), for example, may be favorably positioned at a distance of about 20 cm or less (including down to 0 (zero) cm) from the second end (e.g. the closed end) of the tube.

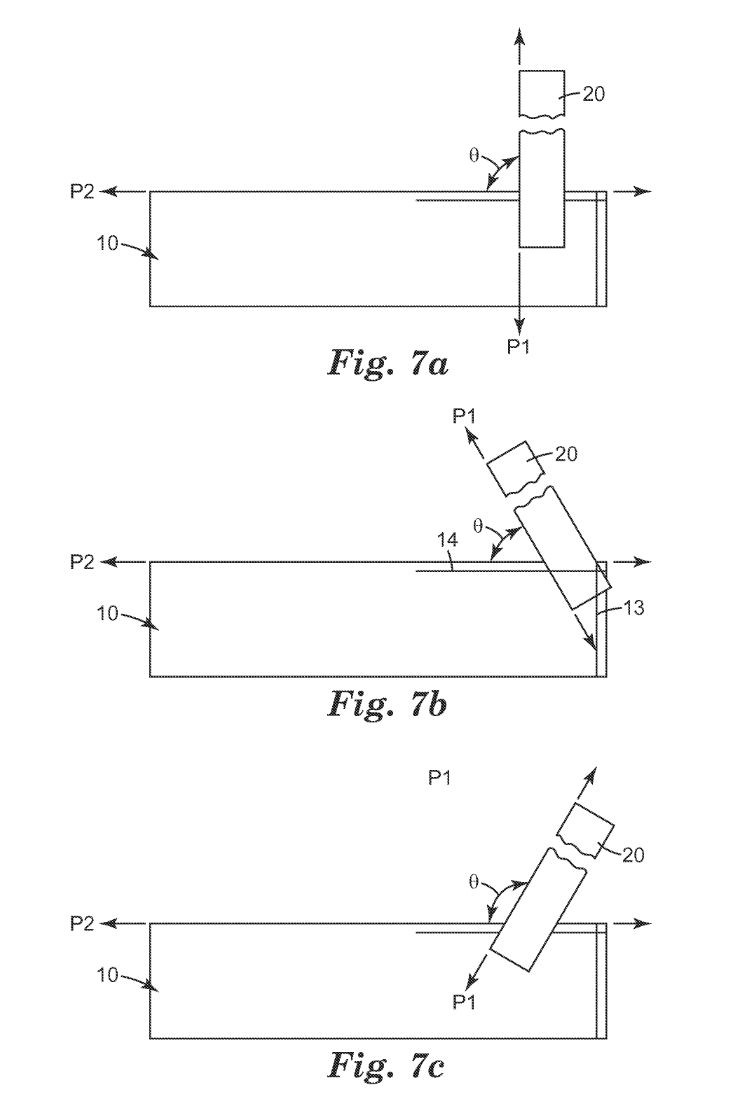

In order to e.g. facilitate ease in draping/wrapping and desirable handleability (e.g. lifting and/or shifting of the extremity while pulling on the elongated bandage) during draping/wrapping, the first end portion of the elongated bandage is permanently affixed to the tube, i.e. affixed to the tube in such a manner that it cannot be detached from the tube under routine use. The first end portion of the elongated bandage may be completely or intermittently or peripherally affixed. Affixing can be accomplished by any suitable method allowing for a permanent affixing of the first end portion of the elongated bandage, including for example thermal bonding or welding, ultrasonic welding, stitching, permanent adhesive bonding. In some cases, affixing of the first end portion of the elongated bandage can be accomplished simultaneously with sealing of appropriate edges or portions of edges of the tube, if as a result of the particular selected positioning of the elongated bandage its first end portion overlays in part such portions of the tube to be sealed and if an appropriate method of affixing/sealing is or can be used. For example, FIG. 7b shows an exemplary embodiment (where the elongated bandage is extended along its length in a single direction to lay flat) with thermal or ultrasonic welding along lines 13 and 14 to allow for provision of seams of the tube (10) together with affixing of the first elongated portion of the elongated bandage (20) to the tube in a single manufacturing operation.

As can be appreciated from the exemplary embodiments shown in FIGS. 1 to 4, the elongated bandage is desirably provided in the form of a roll. The elongated bandage may be rolled on itself or rolled up on a roll core. For example, referring to FIG. 5 showing a cross-section of the exemplary embodiments shown in FIGS. 1 and 3, it can be seen that the elongated bandage is wound on a roll core (22).

Favorably the elongated bandage is provided in an orientation such that it can be easily wrapped around the extremity being draped. Referring a hypothetical state of the elongated bandage being extended flat along its length in a single direction, a plane tangential to a major longitudinal edge of the elongated bandage is favorably perpendicular to or substantially perpendicular (e.g. ±5°) a second plane tangential to a major longitudinal edge of the tube. This is best understood by reference to FIG. 7a. FIG. 7a shows the exemplary embodiment of FIG. 3 with the elongated bandage (20) lying flat extended in a single direction, where a plane (P1) tangential to a major longitudinal edge of the elongated bandage is perpendicular to a plane (P2) tangential to a major longitudinal edge of the tube (10), thus defining an angle (θ) of 90°. It will be appreciated that when the two planes (P1, P2) are substantially perpendicular to one another, the angle (θ) will be either less than 90°, down to and including 85°, or greater than 90°, up to and including 95°. In alternative embodiments the elongated bandage may be favorably positioned and oriented at an acute angle or a more acute angle relative to the open end of the tube, where the aforesaid planes define an angle from about 45° to about 85°. This is best understood by reference to FIG. 7b. FIG. 7b shows an alternative embodiment where the elongated bandage is oriented at an acute angle towards the open end of the tube, so that two respective planes (P1, P2) define an angle (θ) of about 60°. Such orientations of the elongated bandage are particularly suitable for example for embodiments where the elongated bandage is positioned adjacent to the second (e.g. closed) end of the tube. In other alternative embodiments, the elongated bandage may be favorably positioned and oriented at an obtuse angle or a more obtuse angle relative to the open end of the tube, where the aforesaid planes define an angle from about 95° to about 135°. This is best understood by reference to FIG. 7c. FIG. 7c shows an alternative embodiment where the elongated bandage is oriented at an obtuse angle relative to (e.g. away from) the open end of the tube, so that two respective planes (P1, P2) define an angle (θ) of about 125°. Such orientations of the elongated bandage are particularly suitable for example, for embodiments where the elongated bandage is positioned at distance from second end of the tube corresponding to the approximate position of the outer major joint (e.g. an ankle or wrist joint) of the extremity to be draped, in particular when it is desired to first wrap down the tube towards the second end of the tube to wrap the outermost portion of the extremity (e.g. the foot or hand) and then subsequently to wrap up towards the open end of the tube

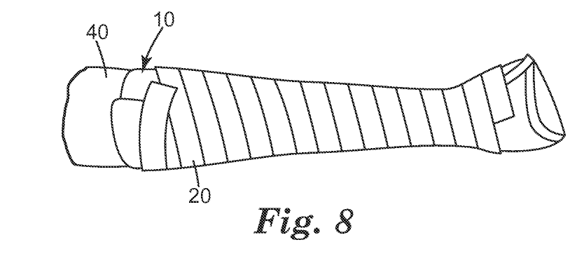

As mentioned above, when using an extremity drape described herein, the extremity is inserted into the tube through the first open end, any excess draping material of the tube in its width can be folded or flapped over, and then the elongated bondage is wrapped about the extremity. In certain preferred embodiments described herein, the elongated bandage is wrapped about the extremity generally in a direction up towards the open end of the tube. For such embodiments, it will be appreciated that depending of the particular, selected positioning of the elongated bandage relative to the second end of the tube, before wrapping up towards the open end of the tube, if desired the elongated bondage may be first wrapped towards to the second end to wrap the outermost portion of the extremity. It will be appreciated that the overall size, e.g. length and width of the elongated bandage can vary, provided it can accommodate the dimensions, e.g. width and length, of the extremity to be draped. For example, for an adult human leg extremity drape the elongated bandage may have a length from about 2 m to about 8 m and a width from about 35 mm to about 150 mm. In regard to the length of the elongated bandage, desirably the length is sufficiently long so that the elongated bandage can be wrapped up to a position near or adjacent to the top edge of the tube when the extremity drape is in place on the patient. This can be best appreciated by reference to FIG. 8 showing a fragmentary view of a patient whose leg (40) is draped with an exemplary extremity drape of the type shown in FIG. 3 where the elongated bandage (20) is wrapped up the tube (10) finishing at a position near the top edge of the tube. It will be appreciated that for adjustment of the length of the tube that is too long for a particular, individual patient, a portion of the tube at its open end can be folded over to provide a cuff overlying the elongated bandage or alternatively the elongated bandage can be wrapped over such a cuff.

For ease in wrapping and/or to provide a close fit, desirably the elongated bandage is an elastic bandage.

After the elongated bandage is wrapped about the extremity, the second end or second end portion thereof may be secured to itself and/or to the tube through the use any suitable fastener, such as a clip or a piece of tape. However for enhanced handleability and ease in use e.g. by one person (e.g. a nurse) as well as enhanced stability of the resulting draping, desirably the means for securing the elongated bandage is provided as an integral element of the extremity drape.

In certain favorable embodiments, at least the second end portion of the elongated bandage is provided with an adhesive. The adhesive can then be used to secure the second end or second end portion of the elongated bandage onto the bandage itself and/or on the outer surface of the tube. Desirably the adhesive is provided on one major surface of the at least second end portion. It will be appreciated that the major surface will typically be that surface which in routine use of extremity drape and its elongated bandage will face the outer surface of the tube. The adhesive may be provided in the form of a continuous or intermittent adhesive strip or patch near or adjacent to the second end of the elongated bandage. Adhesive may be provided only in the second end portion of the elongated bandage or provided continuously or intermittently on one major surface of the elongated bandage along substantially the entire length of the elongated bandage. The latter embodiments allow for a desirable securing of the elongated bandage to itself and to the outer surface of the tube along substantially the entire length of the bandage as it is wrapped up the tube, and thus further assuring desirable stability of the extremity drape. The adhesive may be covered with a protective release liner. However for ease in use desirably the adhesive is protected, prior to use, by providing the elongated bandage in the form of a roll, where adhesive provided in the second end portion may be suitably protected through the use of a roll core, and if applicable, any adhesive provided outside the region of the second end portion may be suitably protected by windings of the bandage on itself where the outer surface of the adhesive will be covered by the opposite major surface of the bandage. Any suitable adhesive may be used. However to facilitate a routine detachment (e.g. from the bandage itself and/or the outer surface of the tube) and unwinding of the elongated bandage, and thus easy removal of the extremity drape, after the completion of an operative procedure, releasable adhesives are favored. Among releasable adhesives, re-positionable adhesives are more favored, because such adhesives allow a re-positioning of the elongated bandage during draping/wrapping, if need be.

Examples of elastic adhesive bandages include bandages commercially available under the trade designation NEXCARE (3M Company, St. Paul, Minn., USA) and PORELAST ((Lohmann & Rauscher GmbH & Co. KG, Neuwied Germany).

In other, more favorable embodiments, at least the second end portion of the elongated bandage is self-adhering. The provision of an elongated bandage where at least the second end portion is self-adhering (also known as self-adherent, self-adhesive or cohesive) is advantageous in that the at least second end portion of the elongated bandage (e.g. at least the last wrap or two of the bandage) can stick to itself and thus securing the bandage. Just the second end portion of the elongated bandage may be self-adherent, or more favorably the elongated bandage is self-adherent over substantially its entire length (e.g. intermittently self-adherent over its entire length) or self-adherent over its entire length. The latter embodiments allow for a desirable securing of the elongated bandage to itself along substantially the entire length or along the entire length of the bandage as it is wrapped up the tube providing a highly stable extremity drape. For enhanced ease in handling during draping/wrapping and detachment after completion of the operative procedure as well as minimization of any risk of damage to the tube element during draping/wrapping, desirably such elongated bandages do not adhere to clothing, hair or skin. Preferred are self-adherent elastomeric bandages, more preferably self-adherent elastomeric bandages that do not adhere to clothing, hair or skin.

Examples of suitable self-adherent elastomeric bandages include bandages commercially available under the trade designation COBAN (3M Company, St. Paul, Minn., USA). Examples of other suitable types of self-adherent elastomeric bandages as well as methods of making such bandages are disclosed in U.S. Pat. Nos. 3,575,782 and 4,984,585 and U.S. Patent Application Publication No. 2005/0025937A contents of which are incorporated in their entirety herein by reference. Examples of other suitable types of self-adherent elastomeric bandages are disclosed in U.S. Pat. No. 6,156,424, content of which is incorporated in their entirety herein by reference. Other examples of suitable types of self-adherent bandages include knitted and woven bandages commercially available under the trade designations ROSIDAL HAFT (Lohmann & Rauscher GmbH & Co. KG, Neuwied Germany) and ACTICO (Activa Health Care, Burton-upon-Trent, UK).

Patient extremity drapes described herein may be reusable or disposable, but preferably they are disposable.

Favorably patient extremity drapes described herein are provided sterilized, folded (e.g. fan-folded) and/or rolled up, and sealed in pouch, thereby ready for use e.g. in an operating room. Two or more extremity drapes may be provided in a single package or alternatively one or more extremity drapes may be provided in a single custom package including other drapes or items for a particular operative procedure. To avoid any potential of waste, typically it is prudent to provide a single extremity drape within a single sealed package. The sealed package may comprise two pouches, an inner sterile pouch containing the drape(s) and an outer non-sterile pouch. Sterilization can be achieved through methods known in the art, such as gamma sterilization or ethylene oxide sterilization, where the selection of the particular sterilization method may in part depend on the particular composition of the relevant elements of the drape. For example, an extremity drape provided as shown in FIG. 3 with a tube having an inner layer made of a nonwoven (e.g. spun-bond) or a nonwoven laminate (e.g. SM or SMS) of polypropylene fibers laminated to a co-extensive, extruded polyethylene outer layer and a self-adherent elastomeric bandage of the type commercially available under the trade designation COBAN can be ethylene oxide sterilized.

1. A drape for covering a patient extremity comprising
a flexible tube having a first open end, a second end and an outer surface,
an elongated bandage having a first end portion,
wherein the first end portion of the elongated bandage is permanently affixed to the tube and wherein said first end portion of the elongated bandage is positioned on the outer surface of the tube.

2. A drape according to claim 1, wherein the second end of the tube is closed.

3. A drape according to claim 1, wherein the first end portion of the elongated bandage is positioned on the outer surface of the tube near or adjacent to the second end of the tube.

4. A drape according to claim 1, wherein the first end portion of elongated bandage is positioned from the second end at a distance equal to or less than that corresponding to the underlying position of the outer major joint of the extremity, when the extremity is inserted into the drape.

5. A drape according to claim 1, wherein the elongated bandage is an elastic bandage.

6. A drape according to claim 1, wherein the elongated bandage has a second end portion and at least the second end portion of the elongated bandage is self-adhering.

7-8. (canceled)

9. A drape according to claim 1, wherein the elongated bandage has a second end portion and at least the second end portion of the elongated bandage is provided with an adhesive, said adhesive being provided on one major surface of the elongated bandage.

10. A drape according to claim 1, wherein when the elongated bandage would be extended flat along its length in single direction,
either
a plane tangential to a major longitudinal edge of the elongated bandage is perpendicular to or substantially perpendicular to a second plane tangential to a major longitudinal edge of the tube;
or
the aforesaid planes define an angle from about 45° to about 85°;
or
the aforesaid planes define an angle from about 95° to about 135°.

11. A drape according to claim 1, wherein the tube includes a slit extending from first open end partially along the length of the tube towards the second end.

12. A drape according to claim 11, wherein the ratio of the length of the tube to the length of the slit is 3:1 or less; more particularly 2:1 or less; most particularly 1.67:1 or less.

13. A drape according to claim 12, wherein the ratio of the length of the tube to the length of the slit is 1.25:1 or more, more particularly 1.33:1 or more; most particularly 1.42:1 or more.

14. A drape according to claim 11, wherein the tube has an inner surface and wherein the inner surface is provided with an adhesive, said adhesive extending along and positioned adjacent to at least a portion of one edge of the slit.

15. A drape according to claim 9, wherein the adhesive is a releaseable adhesive.

16. A drape according to claim 1, wherein the tube is inelastic or substantially inelastic.

17. (canceled)

18. A drape according to claim 1, wherein the tube comprises a liquid impervious material or a nonwoven

19. A drape according to claim 1, wherein the tube is made of a composite material comprising at least two layers.

20. (canceled)

21. A drape according to claim 18, wherein the tube comprises a nonwoven-containing laminate selected from the group consisting of SM, SMS, SMMS, SF, and SFS.

22. (canceled)

23. A drape according to claim 1, wherein the drape is disposable.

24. A drape according to claim 1, wherein the drape does not comprise an integral, elastic, knitted stockinette.

25. A drape according to claim 1, wherein the drape is free of a traction strip mounted within the tube.
